(19) United States
(12) Patent Application Publication
(10) Pub. No.: US 2007/0016149 A1
(43) Pub. Date: Jan. 18, 2007

(54) Injection Needle

(76) Inventors: Marcel Hunn, Langenthal (CH); Susanne Barkhahn, Bern (CH); Andreas Reinmann, Luterbach (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/456,652

(22) Filed: Jul. 11, 2006

Related U.S. Application Data
(63) Continuation of application No. PCT/EP05/00317, filed on Jan. 14, 2005.

(30) Foreign Application Priority Data
Jan. 16, 2004 (DE) 10 2004 002 472.3

(51) Int. Cl. A61M 5/32

(52) U.S. Cl. 604/272

(57) ABSTRACT

An injection needle for introducing a product into a human or animal body, the needle including a distal needle section with a needle point and a proximal needle section, both sections formed along the injection needle such that the proximal needle section must penetrate the skin to introduce the product, the distal needle section having greater flexural rigidity than the proximal needle section.

### CROSS-REFERENCE TO RELATED APPLICATION(S)

This application is a continuation of International Application No. PCT/EP2005/000317, filed Jan. 14, 2005, which claims priority to German Application No. 10 2004 002 472.3, filed Jan. 16, 2004, the entire contents of both of which are incorporated herein by reference.

### BACKGROUND

The present invention relates to devices for delivering, dispensing, administering or injecting a substance, and to methods of making and using such devices. More particularly, it relates to an injection needle for introducing or administering a substance or product into organic tissue, to the use of an injection needle in an infusion set, perfusion set or other delivery device, and to an injection needle assembly in which the injection needle is guided axially. The administration can be made into the skin, subcutaneously, into deeper-lying tissue layers, for example into muscle tissue, or intravenously.

One form of administration of medical or cosmetic products, or of health care products in general, is the subcutaneous administration by a cannula. An example of subcutaneous administration of a product is the administration of insulin in diabetes treatment. For this administration, a cannula tip is positioned subcutaneously. For this purpose, the cannula has to be guided through the skin. Steel cannulas are generally used that have a sufficient flexural rigidity for piercing and penetrating the skin. Because of the required flexural rigidity, however, these injection cannulas are inflexible during the administration and are therefore a source of discomfort, especially if the cannula remains in the body tissue over a considerable period of time. For cannulas remaining at the injection site, infusion sets are known which comprise a catheter head whose inlet end is connected to a catheter that delivers the product to be administered, and whose outlet end comprises the injection cannula. The lack of flexibility of the injection cannula is a disadvantage of these infusion sets in particular.

To overcome this disadvantage, infusion sets are known that comprise a cannula which remains in the tissue during the administration and which is so flexible that it does not cause discomfort when implanted. Because of their flexibility, however, the flexible cannulas cannot be introduced into the tissue without supporting aids. They would bend or even buckle if an attempt was made to insert them into the skin. To remedy this drawback, steel needles are generally used. The steel needle extends through the flexible cannula and protrudes from the distal or free end thereof. The flexible cannula bears tightly on the steel needle so that it can be pushed together with the steel needle into and through the skin and in this way can be positioned subcutaneously. After the subcutaneous positioning, the steel needle is withdrawn from the cannula, and the cannula remains in the tissue for administration. However, such systems require additional work for sealing after withdrawal of the steel needle and also necessitate careful removal of air. Therefore, their handling is awkward compared to simple injection cannulas made of steel and they are more prone to malfunctioning.

### SUMMARY

An object of the present invention to make available an injection needle for introducing a product into organic tissue, which needle is easy to handle when introducing it into the tissue, and flexible within the tissue.

In one embodiment, the present invention comprises an injection needle for introducing a product into a human or animal body, the needle comprising a distal needle section with a needle point and a proximal needle section, both sections formed along the injection needle such that the proximal needle section must penetrate the skin to introduce the product, the distal needle section having greater flexural rigidity than the proximal needle section.

In one embodiment, an injection needle according to the present invention can be pushed as such into and through the skin and remains in the tissue with a flexibility that is sufficient to ensure that the injection needle is not a source of discomfort after its introduction into the tissue. The injection needle comprises a distal needle portion (which also may be referred to as the free or front portion) which extends as far as and comprises a tip of the injection needle, and a proximal needle portion (which also may be referred to as the rear portion). These two needle portions are formed along the longitudinal axis of the injection needle in such a way that the proximal needle portion has to be pushed into the skin for the injection needle to reach the desired depth. The injection needle is composed only of the two needle portions.

According to the invention, the distal needle portion has a greater flexural rigidity than the proximal needle portion. The proximal needle portion forms a flexible site at least locally, but in some embodiments along its entire length. The distal needle portion of greater flexural rigidity lying deeper in the tissue therefore causes no discomfort, or causes much less discomfort than in the case of the known steel cannulas. Of course, the proximal needle portion still has to have sufficient stability to ensure that it does not become completely or largely constricted in the tissue, for example by buckling, such that proper administration of the product is then no longer guaranteed. However, the proximal needle portion can have a flexural rigidity that is as low as the flexural rigidity of the known flexible cannulas.

About its outer circumference, the injection needle can form one or more outwardly open channels in which the product is introduced into the tissue. In such designs, the injection needle is not hollow, or it additionally has an inner lumen for the product to pass through. However, in some preferred embodiments, the injection needle is an injection cannula, i.e. a hollow injection needle with at least one, in some cases, exactly one, inner lumen through which the product is introduced into the tissue. Where the following text refers to an injection cannula instead of an injection needle and refers not to a needle but instead to a cannula or cannula portions, the intent is to describe any structure suitable for delivering or adminsistering a substances, e.g., a needle, cannula, catheter, conduit, tube, etc, including non-hollow injection needles which have one or more outwardly open channels on an exterior surface for guiding the product.

In some preferred embodiments, the distal needle portion is made of a material having a greater modulus of elasticity than the material from which the proximal needle portion is made. The material forming the distal needle portion is therefore harder. The greater the modulus of elasticity of the material, the thinner it is possible to make the cannula tip and/or the sharper the distal edge of the distal needle portion, as a result of which the forces that lead to bending or even to buckling during insertion into and through the skin are reduced.

The flexural rigidity is the product of modulus of elasticity and geometrical moment of inertia. The greater flexural rigidity can therefore in principle also be achieved through a greater geometrical moment of inertia of the distal needle portion compared to the proximal needle portion. However, with an increased geometrical moment of inertia, the modulus of elasticity of the material forming the distal needle portion should not be less than the modulus of elasticity of the material forming the proximal needle portion.

In some preferred embodiments, the material forming the distal needle portion is a composite material. When using a composite material, the statements made above concerning the modulus of elasticity may also apply for the composite. It is clear, however, that at least one of the materials forming the composite must as such have a greater modulus of elasticity than the material forming the proximal needle portion, in which case the material forming the proximal needle portion can also be a composite material. Thus, for example, a first material can be a support material for embedded strengthening elements which as such have a greater modulus of elasticity than the material forming the proximal needle portion. If the material forming the proximal needle portion and the support material are the same materials, the composite forming the distal needle portion already has the greater modulus of elasticity on account of the embedded fibers.

One preferred composite material for the distal needle portion is formed from a support material and a coating with which the support material is coated on its inner circumferential surface or its outer circumferential surface. If appropriate, the support material can be provided with a coating both on the inside and on the outside, or a suitable material can be integrated into the support material. The coating is composed of a material having such a high modulus of elasticity that the composite of support material and coating in each case has a higher modulus of elasticity than the material that forms the proximal needle portion. The coating can be formed by means of a liquid which is dried on the support material and thus hardened. It forms, as it were, a kind of paint. The coating can be homogeneous and, as such, have the sufficiently high modulus of elasticity. However, it too can already be a composite material with embedded strengthening elements, for example fibers, oriented in the longitudinal direction of the injection needle. Instead of fibers, or in addition to fibers, it is also possible for hard grains to be embedded in the coating. For this purpose, a powder or finely particulate grains can be mixed in finely distributed form into a coating liquid.

In some preferred embodiments, the proximal needle portion is formed by an elastically resilient base needle which, in the distal needle portion, is strengthened to increase the flexural rigidity. This strengthening may be achieved by coating the base needle, as has been described above; the base needle forms the support material.

The greater flexural rigidity in the distal needle portion can be obtained not only by means of a coating, but in principle also by the thickening of the distal needle portion made as a whole from a material including composite material with a higher modulus of elasticity than the material forming the proximal needle portion. In such cases too, support materials with embedded or integral strengthening elements, fibers and/or hard grains, can be used as composite materials.

In another embodiment, a base material that can be converted by a chemical reaction, for example a cross-linking reaction of a plastic, into a harder material forms the proximal needle portion, and the converted, harder material forms the distal needle portion. Directly during the forming of the needle or after its forming, the distal needle portion that includes the needle tip goes through a process step effecting the reaction, whereas the more flexible distal needle portion is not converted, to maintain the greater flexibility desired there.

Finally, the greater flexural strength can also be achieved by insertion or attachment of a solid sleeve which forms the cannula tip and which is made of a flexurally rigid, hard material, for example steel. The sleeve can be inserted into a base needle already forming the proximal needle portion as such or can be fitted over it.

In some embodiments, the proximal needle portion should be longer than the distal portion. In one preferred embodiment, it is at least twice as long as the distal needle portion.

The following are exemplary tolerances or specifications of some embodiments of the present invention:

the distal needle portion should have a length of at least approximately 0.5 mm, measured from the needle tip. It is at most approximately 8 mm in length. If the injection needle is inserted deeper than is customary for subcutaneous administration, for example for intravenous administration, then the values for the preferred minimum length and maximum length change proportionally according to the total length of the injection needle.

the modulus of elasticity of the material from which the distal needle portion is made should be at least approximately 1000 MPa-3000 MPa.

the proximal needle portion should have a length of at least approximately 2 mm.

the modulus of elasticity of the material from which the proximal needle portion is made is less than approxiamately 3000 MPa-2000 MPa, but should be greater than approximately 500 MPa-1000 MPa.

A catheter through which the product to be administered is delivered to the injection needle can form the proximal needle portion in one piece. If appropriate, a transition is formed by the catheter thinning from a greater catheter cross section to a smaller cannula cross section.

In some preferred embodiments, the injection needle is part of an infusion set. The infusion set comprises a housing with an underside which can be positioned on the skin at the injection site. Although, during use, the housing can in principle be held at the injection site by means of the injection needle inserted into the tissue, in some preferred embodiments, the underside of the housing is itself made ready for fixing to the injection site. This can be achieved, for example, by an adhesive pad located on the underside, as in conventional infusion sets. The injection needle is supported by a holding part of the housing and protrudes from an underside of the holding part. The infusion set further comprises a catheter for delivery of the administered product to the housing, and a fluid connection which is formed in or on the housing and which serves to connect the catheter to the injection needle. The fluid connection can be formed with the catheter in one piece. The upstream end of the connecting line can be connected to the catheter by means of a quick-coupling mechanism formed by the housing and is able to be detached again from the catheter. The fluid connection is permanently connected to the injection needle.

In one preferred infusion set in accordance with the present invention, the housing guides the injection needle in an axial movement and stabilizes it against buckling and bending during the axial movement. In these embodiments, the housing forms a needle guide. The needle guide thus provides lateral support for the injection needle. In a preferred embodiment, the needle guide can be axially shortened to permit insertion of the needle into the skin. In such a configuration, the needle guide can be axially shortened in an elastic or permanent manner. The axial shortening can be achieved by designing the needle guide so that it can collapse or fold up. A collapsible needle guide can be formed as a balloon or as a porous structure. A bellows structure can, for example, form a foldable needle guide.

In some embodiments, when a pressure is applied to the skin, the housing tensions the skin before injection, and the pressure force needed to insert the needle into the skin is reduced. The guiding and stabilizing may be thought of as separate, distinct functions or features, but they may be applied in combination, i.e. the housing then forms a needle guide that tensions the skin.

In other preferred embodiments, the injection needle is part of a perfusion set. Perfusion sets can be used in particular for diagnostic purposes, for example for determining the glucose content in a body fluid, for example in diabetes therapy. By means of such perfusion sets, body fluid is transported out of the body by means of a flushing liquid, comparable with the flushing in dialysis, and is delivered to a sensor, in this example a glucose sensor. The statements made herein concerning infusion sets apply equally to perfusion sets. A part of such sets that can be positioned on body tissue forms a needle injection unit comprising the injection needle and the needle guide.

### BRIEF DESCRIPTION OF THE DRAWINGS

FIG. 1 shows an injection needle according to one illustrative embodiment of the present invention,

FIG. 2 shows a cross section through a distal needle portion of the injection needle,

FIG. 3 shows a cross section through a needle portion of an injection needle according to another illustrative embodiment,

FIG. 4 shows a cross portion through a needle portion of an injection needle according to another embodiment,

FIG. 5 shows an injection needle according to another illustrative embodiment in a longitudinal section,

FIG. 6 shows another embodiment of the present invention comprising a catheter head and an injection needle,

FIG. 7 shows the catheter head at an injection site before the injection needle is inserted into the skin,

FIG. 8 shows the catheter head before the injection, but with pressure applied,

FIG. 9 shows the catheter head after the injection needle has been inserted into the skin,

FIG. 10 shows a catheter head according to another illustrative embodiment,

FIG. 11 shows the catheter head of FIG. 10 after the injection needle has been inserted into the skin,

FIG. 12 shows a catheter head according to another illustrative embodiment with an injection needle,

FIG. 13 shows the catheter head of the embodiment of FIG. 12, and

FIG. 14 shows the catheter head of embodiment of FIG. 12 after the injection needle has been inserted into the skin.

### DETAILED DESCRIPTION

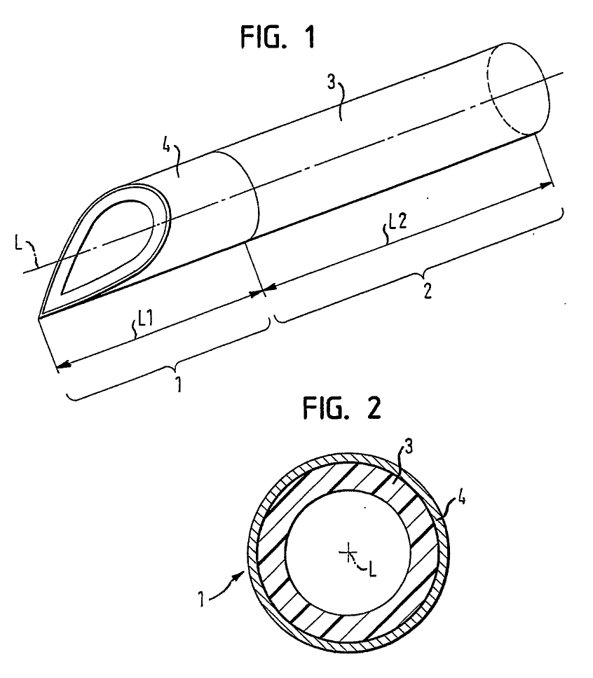

FIG. 1 shows an illustrative embodiment of the present invention comprising an injection needle in the form of an injection cannula with two different cannula portions. The cannula portions include a distal cannula portion 1, which forms a free tip of the injection cannula, and a proximal cannula portion 2 which adjoins the distal cannula portion 1. A transition between the cannula portions 1 and 2 is substantially linear in a cross-sectional surface perpendicular to a longitudinal axis L of the injection cannula.

The distal cannula portion 1 has a length L1 measured along the longitudinal axis L, and the proximal cannula portion 2 has a length L2. The sum of the lengths L1 and L2 corresponds to the length of conventional injection cannulas for subcutaneous administration of a medicament, for example insulin, and it is for this purpose that the injection cannula according to the invention is also employed. The total length L1+L2 of the injection cannula thus amounts to between 4 and 16 mm. The table below shows approximate lengths for these injection cannulas, the data regarding L1 being shown with approximate upper and lower limit values, and the difference to L1+L2 being compensated by L2:

| L1 + L2 | L1 |
|---|---|
| 4 mm | 0.1-2 mm |
| 6 mm | 1-3 mm |
| 8 mm | 1-4 mm |
| 10 mm | 1-5 mm |
| 12 mm | 1-6 mm |
| 14 mm | 1-7 mm |
| 16 mm | 1-8 mm |

In some embodiments, the shape of the injection cannula generally corresponds to that of conventional injection cannulas, i.e. it is circular and has a circular hollow cross section. The distal cannula portion 1 is beveled to form the tip. The proximal cannula portion 2 is made of a plastic material. Possible plastic materials in question are all suitable materials, including those that are used in conventional, flexible infusion cannulas. Polymer acrylate is one example.

The distal cannula portion 1 is composed of a two-layer composite material. The inner of the two layers is composed of the same material as the proximal cannula portion. The inner layer of the distal cannula portion 1 and the proximal cannula portion 2 form a one-piece base cannula 3. To obtain greater flexural rigidity in the distal cannula portion 1, the base cannula 3 in the distal cannula portion 1 is provided with an outer coating 4. The outer coating 4 is applied uniformly on the outer circumferential face of the base cannula 3. Its thickness is much smaller than the thickness of the base cannula 3. The coating 4 is formed as a hard lacquer layer which also covers the tip of the injection cannula. The modulus of elasticity of the coating 4 should be at least twice as great as the modulus of elasticity of the material of the base cannula 3. The combination of base cannula 3 and coating 4 has a greater overall flexural rigidity than the base cannula 3 and, therefore, than the proximal cannula portion 2. This is related in each case to the annular cross section per portion 1 and 2. The coating 4 is formed by application of a liquid that hardens or is hardened after application. The coating 4 can be applied to the inside and outside, for example by immersing the cannula portion 1 in an immersion bath. To obtain the coating 4 with a small layer thickness, the applied liquid has a low viscosity, so that the coating 4 is like a paint.

FIG. 2 shows the distal cannula portion 1 of the injection cannula from FIG. 1 in a cross section. The wall thickness of the base cannula 3 can correspond to the wall thickness of conventional flexible infusion cannulas. The thickness of the coating 4 amounts to approximately 10% or less of the thickness of the base cannula 3.

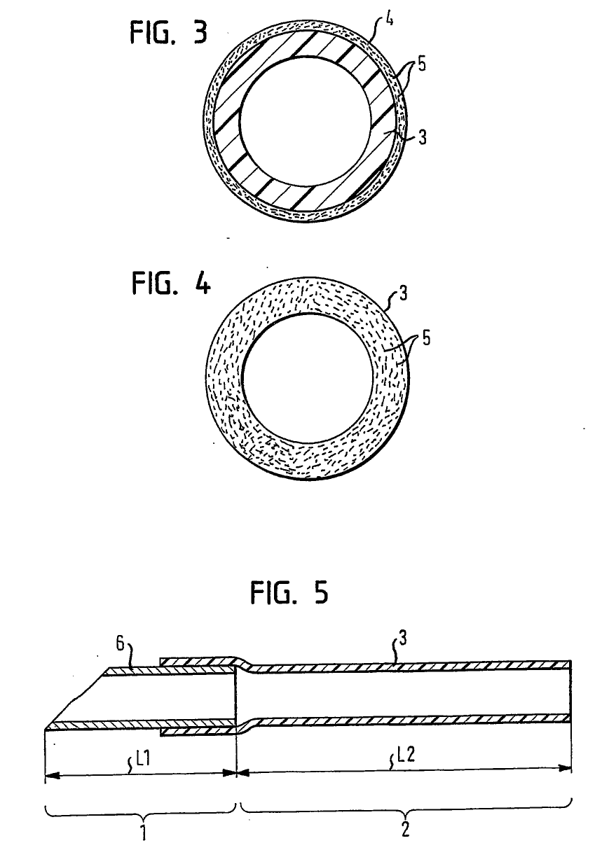

FIG. 3 shows an injection cannula in another illustrative embodiment, again in cross section through its distal cannula portion 1. The proximal cannula portion 2 of the injection cannula in the second illustrative embodiment corresponds to the proximal cannula portion 2 of the first illustrative embodiment. The cannula portion 1 of the second illustrative embodiment differs from that of the first illustrative embodiment only in terms of a modified coating 4. In contrast to the coating 4 of the first illustrative embodiment, which is composed homogeneously of a hardened coating material, the coating 4 of the second illustrative embodiment has longitudinal fibers 5 embedded or integrated into the same support material, these fibers 5 further increasing the modulus of elasticity of the coating 4 and therefore also the modulus of elasticity of the combination of base cannula 3 and coating 4 relative to the first illustrative embodiment. With the same cross-sectional shape and surface area as in the first illustrative embodiment, the flexural rigidity as a product of the modulus of elasticity and geometrical moment of inertia is correspondingly greater.

Another illustrative embodiment of an injection cannula is shown in FIG. 4. Again, only a cross section through the distal cannula portion 1 of the third illustrative embodiment is shown. The cannula portion 1 of this embodiment is likewise composed of a composite material, which however consists of only one layer of the material of the base cannula 3 of the first and second illustrative embodiment and of longitudinal fibers 5 embedded therein. Unlike in the second illustrative embodiment, the longitudinal fibers 5 are thus not embedded only in a coating, but directly in the whole cross-sectional surface area of the base cannula 3, which forms the proximal cannula portion 2, too.

Instead of or in addition to the longitudinal fibers 5, granular particles can be embedded in the coating 4 or in the base cannula 3 in the distal cannula portion 1, these particles also leading to an increase in the modulus of elasticity compared to the material of the base cannula 3. If appropriate, the distal cannula portion 1 can also be made up of more than two concentric layers. It is also possible to apply a coating which works into the material of the base cannula 3 and there leads to an increased modulus of elasticity across the entire cross-sectional area or at least in an outer part of the cross-sectional area.

FIG. 5 shows an injection cannula according to a fourth illustrative embodiment of the present invention. A base cannula 3, which is sufficiently flexible (like the base cannula 3 of the other illustrative embodiments) so as not to cause discomfort in the inserted state, but which on the other hand is sufficiently stable to ensure an adequate cross section of flow for product administration despite the desired resiliency and the associated deformation, extends along almost the entire length L1+L2 of the injection cannula. To obtain the greater flexural rigidity in the distal cannula portion 1, a thin sleeve 6 is inserted into the base cannula 3, the length of which sleeve 6 is intended to correspond to the lengths L1, as indicated in the first illustrative embodiment, plus an additional length of approximately 5 to 20%. The sleeve 6 is pressed into the base cannula 2 so that the base cannula 3 is tensioned around the sleeve 6. The hollow cross section of the sleeve 6 generally corresponds to the hollow cross section of the unloaded base cannula 3. The sleeve 6 protrudes from the base cannula 3 by the stated additional length and forms the cannula tip. This results in an injection cannula being obtained which is somewhat reminiscent of the conventional systems with a flexible cannula and with a steel needle extending through the latter. However, the base cannula 3 and the sleeve 6 are fixedly connected to one another and form one unit. During administration of the product, the sleeve 6 remains in the base cannula 3 and is also discarded together with the latter after use, such that handling is made much simpler compared to the known, two-part systems.

The sleeve 6 can be a steel sleeve and correspond to a short portion of conventional steel cannulas for subcutaneous administration of products. Instead of an inner sleeve, the injection cannula can also be formed with an outer sleeve.

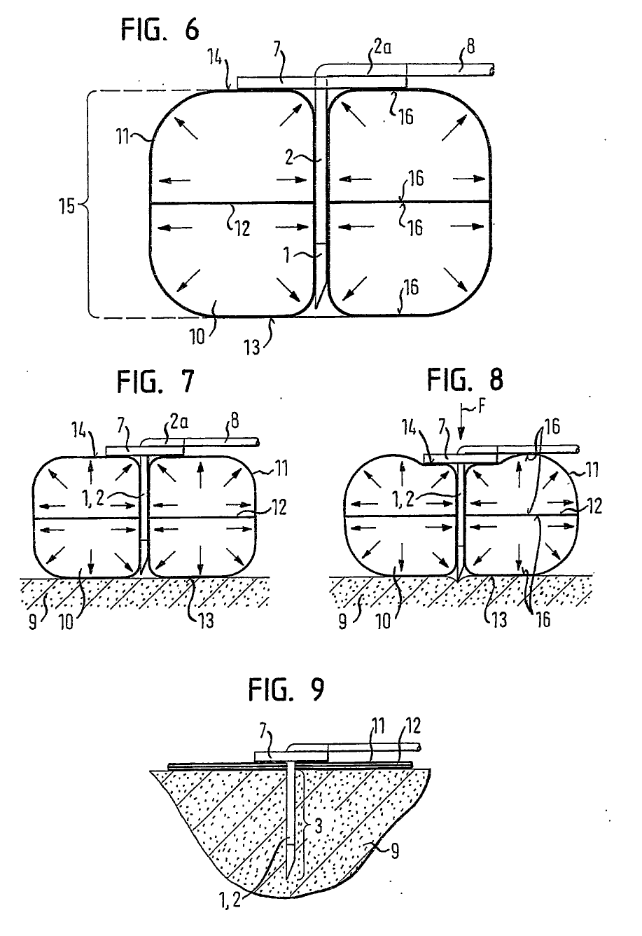

FIGS. 6 to 14 show cannula units which are obtained with an injection cannula according to the present invention and which are in the form of catheter heads of infusion sets, for example an infusion set for administration of insulin. Such infusion sets may be used for self-administration, i.e. administration to oneself. The catheter head guides the injection cannula in axial movement and supports it laterally, such that the injection cannula is stabilized against bending and buckling when pressed into and through the skin.

FIG. 6 shows, in a first illustrative embodiment, a cannula unit comprising an injection cannula with portions 1 and 2, a cannula guide 10 for the injection cannula, and a pressure force distributor 7. The cannula unit serves for subcutaneous administration of a liquid product, a medicament, for example insulin. With its proximal end, the cannula portion 2 forms a securing portion 2a which is at an angle, in the illustrative embodiment at a right angle, to the distal part of the cannula portion 2. The securing portion 2a is connected to a catheter 8 for delivery of the product. The pressure force distributor 7 has a planar configuration, in the form of a round plate in the illustrative embodiment.

The injection cannula and the pressure force distributor 7 are separately produced parts. The injection cannula is held with frictional engagement in the central passage of the pressure force distributor 7 and is secured lying flat on the top face of the pressure force distributor 7. In a modified design, the injection cannula and the pressure force distributor 7 can also be formed in one piece, or the injection cannula can be embedded with its securing portion 2a in the pressure force distributor 7 and cohesively connected to the pressure force distributor 7.

The cannula guide 10 is an air-filled balloon with a flexible balloon wall 11, so that a cannula guide is obtained which has a flexible axial portion 15 between an underside 13 and a top face 14. The balloon 10 is annular and encloses the injection cannula. The cannula tip is set back a short distance behind an underside 13 of the balloon 10. The pressure force distributor 7 is secured lying on the top face 14 of the balloon 10. The balloon 10 bears with its internal pressure uniformly on the injection site. The internal pressure of the balloon 10 is at least as great as the atmospheric pressure, and an overpressure prevails inside the balloon wall 11.

Arranged in the balloon 10 there is a support structure 12, approximately at the axial center of the injection cannula. The support structure 12 is, as the name is intended to suggest, planar and flat in the axial direction, i.e. in the longitudinal direction of the injection cannula. In the illustrative embodiment, the support structure 12 is a thin support plate, a support membrane, which can be deformed into a flat shell. The support structure 12 extends, transversely with respect to the injection cannula 1, across the entire radial width of the balloon 10, from its annular outside wall to its annular inside wall and thus forms, in addition to the annular inside wall of the balloon 10, a local support for the injection cannula.

The underside 13 of the balloon 10 is provided, for example coated, with an adhesive, so that an outer adhesive surface is obtained which ensures an adhesive connection of the cannula unit 10 to the surface of the body tissue, generally the surface of the skin. The balloon wall 11 is likewise provided with an adhesive across its entire inner surface. Similarly, the support structure 12 is provided with an adhesive on its underside directed toward the underside 13 and on its top face directed toward the top face 14. In this way, inner adhesive surfaces 16 are obtained which adhere to one another in a collapsed state of the balloon 10. It would in principle suffice to provide an adhesive only on the underside and top face of the support structure 12 and/or only on the inner surfaces of the balloon wall 11 on the underside 13 and top face 14 of the balloon 10.

FIGS. 7, 8 and 9 show the cannula unit of the first illustrative embodiment in use.

In FIG. 7, the cannula unit is placed on the surface of the body tissue 9 and fixed adhesively by means of its underside 13 formed as an outer adhesive surface. No external force is applied to the cannula unit, or at most a light pressure force which is directed axially in the direction of the surface of the body tissue 9 and which is sufficient to establish the adhesive connection. The cannula tip is located a short distance above the surface of the body tissue 9, i.e. there is still no contact with the body tissue 9.

FIG. 8 shows the cannula unit of the first illustrative embodiment in the initial phase of insertion of the injection cannula into the skin. By means of a pressure force F exerted on the pressure force distributor 7 in axial continuation of the injection cannula and directed axially in the direction of the body tissue 9, the pressure force distributor 7 presses against the balloon 10 via the top face 14 of said balloon 10, and the latter accordingly deforms under the pressure force F. Because of the pressure force F, the injection cannula moves axially in the direction toward the surface of the body tissue 9, comes into contact with the surface and initially just presses against the surface, until the surface has reached a critical tension at which the cannula tip pierces the surface and penetrates into the body tissue 9. FIG. 8 shows the cannula unit directly before it pierces the surface of the body tissue 9.

During the movement toward the surface of the body tissue 9, during the piercing of the surface and during the penetration into the body tissue 9, the injection cannula slides along the inside wall of the balloon 10 surrounding it. The support structure 12 stabilizes and guides the injection cannula in the first instance. The balloon 10, in which the support structure 12 is accommodated, additionally supports and guides the injection cannula throughout the entire injection procedure. The support structure 12 and the balloon 10 thus stabilize the cannula portion 2 particularly against bending or even buckling. The injection cannula protruding freely from the underside of the pressure force distributor 7 can therefore have less flexural rigidity, namely a lower modulus of elasticity and/or a lower geometrical moment of inertia, than injection cannulas which are not laterally supported during the piercing of the tissue surface and their onward penetration into the tissue. The injection cannula is accordingly less “bulky” when it is sitting in the body tissue 9 during the administration of product.

The balloon 10 is constructed such that it bursts when its internal pressure exceeds a predetermined limit value. This limit value is provided for through a suitable dimensioning of the balloon wall 11, i.e. through the use of a suitable wall material and through the wall thickness. The balloon wall 11 is configured such that, when the pressure limit value is exceeded, it tears and the balloon 10 suddenly collapses. The design of the balloon 10 is advantageously such that the balloon 10 bursts after the cannula tip is already pressing against the body tissue 9 but when the cannula tip has not yet penetrated the body tissue 9. The penetration, i.e. piercing of the tissue surface, takes place directly upon collapse of the balloon 10.

The balloon 10, and the cannula guide according to the present invention in general, may also be advantageously configured in such a way that, by manual pressure on the top face 14, i.e. the application of the pressure force F, the surface of the body tissue 9 is tensioned at the injection site and, in this way, the pressure force required for penetration of the surface is reduced.

FIG. 9 shows the cannula unit in the implanted state. The injection cannula protrudes with its portions 1 and 2 into the body tissue 9. The balloon 10 has completely collapsed and forms a flat plaster adhering to the surface of the body tissue 9, since the outer adhesive surface on the underside 13 of the previous balloon 10 adheres to the body tissue 9 and the inner surfaces 16 adhere to one another. In this state, a medicinal product may be administered through the injection cannula over the course of several days.

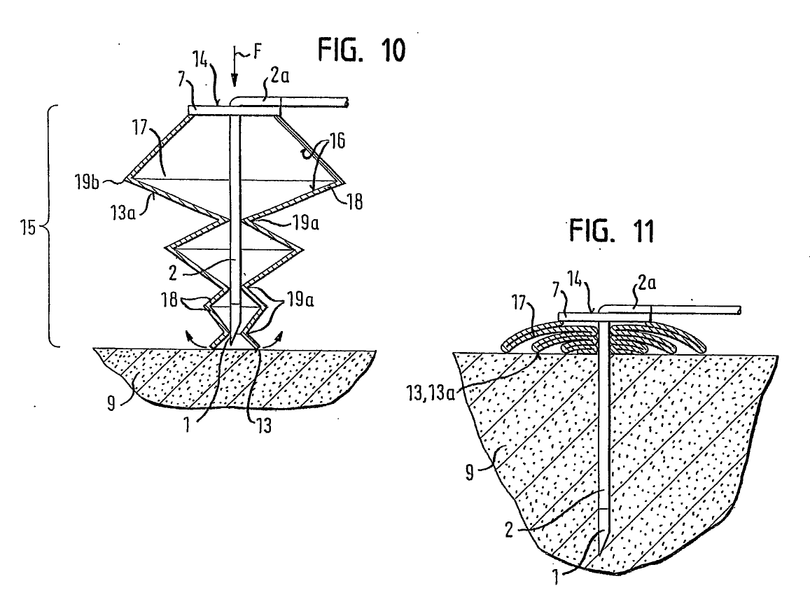

FIG. 10 shows a second illustrative embodiment of a cannula unit consisting of an injection cannula, a pressure force distributor 7 and a cannula guide 17. The injection cannula and the pressure force distributor 7 are designed as in the first illustrative embodiment. The cannula guide 17 also forms a flexible axial portion 15 which, as before in the first illustrative embodiment, extends from the underside 13 to the top face 14 of the cannula guide 17. The cannula guide 17 of the second illustrative embodiment is designed as a bellows with pairs of support webs 18 pointing at an angle to one another and to the cannula portions 1 and 2, and folding joints 19a and 19b which are in each case formed between two adjacent support webs 18. The inner folding joints 19a are not only joints, but at the same time also form a supporting and guiding position for the injection cannula.

The support webs 18 are of different lengths, with the length increasing from the underside 13 to the top face 14. Two support webs 18 of identical or substantially identical length are in each case connected to one another in a foldable manner at the outer folding joints 19b. When the unit is placed in position on the surface of the body tissue 9, the most distal support web 18 points obliquely and radially outward from the most distal inner folding joint 19a, such that an open funnel is obtained on the underside 13. Therefore, as in the first illustrative embodiment, when a pressure force F is exerted, the tissue surface is tensioned at the injection site and, this way, penetration of the tissue surface is made easier.

The bellows structure forming the cannula guide 17 elastically yields in the axial direction when an axial pressure force F is exerted, up to the point where a limit value is reached for the axial pressure force F, but abruptly collapses when the limit value is exceeded. The cannula guide 17 is designed like the cannula guide 10 of the first illustrative embodiment in terms of its deformation properties, as far as the elastic resiliency and abrupt collapse are concerned.

FIG. 11 shows the cannula unit of the second illustrative embodiment in the implanted state of the injection cannula, in which the latter's penetrating portion 3 has penetrated completely into the body tissue 9. In this state, the cannula guide 17 of the second illustrative embodiment likewise forms a flat plaster, because the support webs 18 are folded in pairs on top of one another. To stabilize the cannula guide 17 in the folded state, the support webs 18 are also provided with inner adhesive surfaces 16. Moreover, those support webs 18 with undersides pointing toward the body tissue 9 are provided with outer adhesive surfaces 13a on these undersides, such that the support webs 18 on the one hand adhere to one another via their outer surfaces and, because the support web lengths increase from distal to proximal, they also adhere directly on the surface of the body tissue.

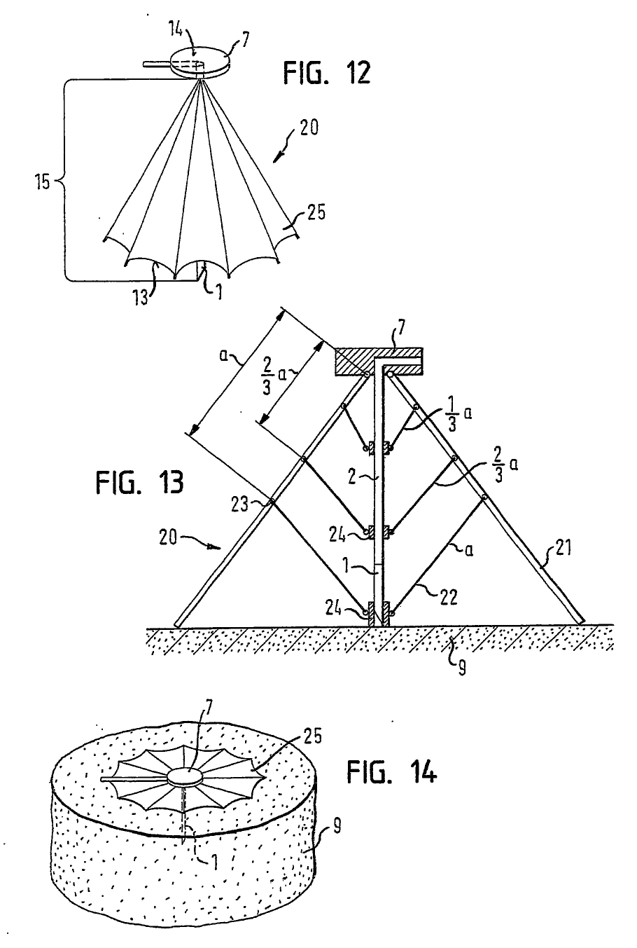

FIG. 12 shows a cannula unit of a third illustrative embodiment. The cannula unit differs from the cannula units of the other illustrative embodiments in terms of its cannula guide 20, which in the third illustrative embodiment is designed as an umbrella structure, i.e. as a structure which can be deployed, opened or spread open in the manner of an umbrella and can thereby be shorted in the length direction of the injection cannula.

FIG. 13 shows the cannula unit of the third illustrative embodiment in a state in which it is placed on the body tissue 9 before insertion of the injection cannula into the skin. As can be seen from FIG. 13, the cannula guide 20 comprises several spreadable struts 21 which are each attached in an articulated manner to an underside of the force distributor 7 directed toward the body tissue 9. The articulated attachment is such that the inherently axially stiff spreadable struts 21 can be pivoted toward the underside of the force distributor 7 at their respective articulation. In relation to the injection cannula, the spreadable struts 21 point radially outward from their articulations. They are arranged in uniform distribution around the injection cannula. The spreadable struts 21 are each supported on the injection cannula via several support struts 22. The support struts 22 are each attached in an articulated manner to the spreadable struts 21 and form an axial slide guide for the injection cannula, which axial guide laterally supports the injection cannula and axially guides it in a linear movement. The articulated attachments of the support struts 22 to the spreadable struts 21 are designated by 23, and the slide guides at the respective other end of the support struts 22 are designated by 24. Along the spreadable struts 21, the articulated attachments 23 are each at a distance from the articulated attachments of the spreadable struts 21 on the force distributor 7 which corresponds to the length of the respective support strut 22. Thus, for example, the support struts 22 which have the greatest distance a from the articulated attachments of the spreadable struts 21 on the force distributor 7 each have a length a corresponding to the distance. The support struts 22 arranged closer to the force distributor 7 each have lengths corresponding to their distances measured along the spreadable struts 21. With uniform distribution, as shown in the illustrative embodiment, lengths ⅔ a and ⅓ a are obtained for the further support struts 22.

FIG. 14 shows the cannula unit of the third illustrative embodiment with the injection cannula inserted into the body tissue 9. The spreadable struts 21 are pivoted, about their articulated attachments on the force distributor 7, toward the force distributor 7 and are thus spread open. The support struts 22 are pivoted about their articulated attachments 23 toward their respective spreadable strut 21 and come to lie one on the other, so that overall a flat structure is obtained in the spread or compressed state, which flat structure at the same time also serves as a plaster for attachment to the tissue surface.

As is indicated in FIG. 12 and can be seen in FIG. 14, the cannula unit of the third illustrative embodiment comprises a plaster 25 which, in accordance with the spreading mechanism, can be designated as an umbrella-type plaster. The plaster 25 is similar to the cover of an umbrella. It is secured on the spreadable struts 21. In the non-inserted state, i.e. before being spread open, it hangs loosely like the cover of an umbrella between the spreadable struts 21, whereas in the inserted state it is stretched out and adheres with its underside on the tissue surface.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection needle for introducing a product into a human or animal body, said injection needle comprising:
a) a distal needle portion with a needle tip, and
b) a proximal needle portion, wherein
c) the portions are formed along the injection needle such that the proximal needle portion has to be pushed into the skin for introducing the product, and wherein
d) the distal needle portion has a greater flexural rigidity than the proximal needle portion.

2. The injection needle as claimed in claim 1, wherein the distal and proximal portions comprise materials, the distal needle portion comprising a material having a greater modulus of elasticity than a material of the proximal needle portion.

3. The injection needle as claimed in claim 1, wherein the distal needle portion comprises a composit material.

4. The injection needle as claimed in claim 1, wherein the proximal needle portion comprises an elastically resilient base needle which, in the distal needle portion, is strengthened to increase the flexural rigidity.

5. The injection needle as claimed in claim 4, wherein the distal needle portion comprises a stiffening element.

6. The injection needle as claimed in claim 4, wherein the base needle in the distal needle portion comprises a stiffening coating.

7. The injection needle as claimed in claim 6, wherein stiffening elements are embedded in the coating.

8. The injection needle as claimed in claim 4, further comprising stiffening elements embedded in the base needle in the distal needle portion.

9. The injection needle as claimed in claim 1, wherein the proximal needle portion comprises a base material convertable by a chemical reaction into a harder material, and wherein the distal needle portion comprises the converted, harder material alone or together with an other material.

10. The injection needle as claimed in claim 9, wherein the base material is a cross-linkable plastic or contains a cross-linkable plastic.

11. The injection needle as claimed in claim 1, wherein the proximal needle portion is formed by an elastically resilient base needle which, in the distal needle portion, is strengthened by an inserted or attached sleeve that increases the flexural rigidity.

12. The injection needle as claimed in claim 1, wherein the distal needle portion has a length of at least 0.5 mm, measured from the needle tip.

13. The injection needle as claimed in claim 1, wherein the distal needle portion has a length of at most 8 mm, measured from the needle tip.

14. The injection needle as claimed in claim 1, wherein the distal needle portion has a length of at least 1 mm and at most 4 mm, measured from the needle tip.

15. The injection needle as claimed in claim 1, wherein the distal needle portion is made from a material that has a modulus of elasticity of at least 2000 MPa.

16. The injection needle as claimed in claim 1, wherein the proximal needle portion has a length of at least 2 mm.

17. The injection needle as claimed in claim 1, wherein the proximal needle portion is longer than the distal needle portion.

18. The injection needle as claimed in claim 1, wherein the proximal needle portion is at least twice as long as the distal needle portion.

19. The injection needle as claimed in claim 1, wherein the proximal needle portion has a modulus of elasticity of less than 1000 MPa.

20. The injection needle as claimed in claim 1, wherein the injection needle is a component of an infusion or perfusion set.

21. An injection needle assembly comprising:
a) an injection needle comprising a distal needle portion with a needle tip and a proximal needle portion, wherein the portions are formed along the injection needle such that the proximal needle portion has to be pushed into the skin for introducing the product, and wherein the distal needle portion has a greater flexural rigidity than the proximal needle portion, and
b) a needle guide which can be placed on a body tissue and axially stabilizes the injection needle against bending and buckling.

22. The injection needle assembly as claimed in claim 21, wherein the needle guide can be axially shortened by an axial pressure exerted to insert the injection needle into the body tissue.

23. The injection needle assembly as claimed in claim 22, wherein an underside of the needle guide is designed such that the skin is tensioned at the injection site by a pressure exerted by the needle guide against the skin.

24. The injection needle assembly as claimed in claim 23, wherein the needle guide forms, on at least two mutually opposite sides of the injection needle, a funnel that widens in the direction of the skin and extends generally around the circumference of the injection needle.

25. An injection needle for introducing a substance into a human or animal body, the needle comprising a first needle section and a second needle section, wherein the second needle section must penetrate the skin of the body to introduce the substance, the first needle section having greater flexural rigidity than the second needle section.
